(19) United States
(12) Patent Application Publication
(10) Pub. No.: US 2025/0120682 A1
(43) Pub. Date: Apr. 17, 2025

(54) INNER CONTAINER OPENING STRUCTURE OF SPERM EXTRACTOR

(71) Applicant: Zhejiang lianhong Technology Co., LTD, Huzhou (CN)

(72) Inventor: Xiu CHEN, Hangzhou (CN)

(73) Assignee: Zhejiang lianhong Technology Co., LTD, Huzhou (CN)

(21) Appl. No.: 18/768,234

(22) Filed: Jul. 10, 2024

(30) Foreign Application Priority Data
Oct. 13, 2023 (CN) 2023227620977

(51) Int. Cl. A61B 10/00; A61H 19/00

(52) U.S. Cl. CPC A61B 10/0058; A61H 19/32

(57) ABSTRACT

Provided in the present utility model is an inner container opening structure of a sperm extractor, including: a soft rubber inner container configured to massage a male genital organ; and a power mechanism mounted on the soft rubber inner container and configured to generate a vibration, where the soft rubber inner container includes an opening portion and a main body portion; the main body portion is mounted at the opening portion, and a channel is formed inside the opening portion and the main body portion; the power mechanism is disposed at the opening portion; a contact plate is disposed at a lower end of the opening portion; the power mechanism is mounted on the contact plate; and two arc-shaped grooves are further formed in the contact plate.

### CROSS-REFERENCE TO RELATED APPLICATIONS

The application claims priority to Chinese patent application No. 2023227620977, filed on Oct. 13, 2023, the entire contents of which are incorporated herein by reference.

### TECHNICAL FIELD

The present utility model relates to the technical field of medical devices, specifically to an inner container opening structure of a sperm extractor.

### BACKGROUND

With the continuous development of economy and society, people's life pressure is also increasing, leading to a decrease in male sperm motility and an increasingly prominent problem of infertility. During clinic services in existing hospitals, it is often required to extract semen in a short time and conduct various tests on the semen.

According to a sperm extractor in the prior art, a vibration mechanism is disposed in the middle of a soft rubber inner container, resulting in a poor vibration effect.

Therefore, the prior art has shortcomings and needs to be further improved.

### SUMMARY

In view of the problems existing in the prior art, the present utility model provides an inner container opening structure of a sperm extractor.

To achieve the above objective, a specific solution of the present utility model is as follows:

The present utility model provides an inner container opening structure of a sperm extractor, including:
a soft rubber inner container configured to massage a male genital organ; and
a power mechanism mounted on the soft rubber inner container and configured to generate a vibration,
where the soft rubber inner container includes an opening portion and a main body portion; the main body portion is mounted at the opening portion, and a channel is formed inside the opening portion and the main body portion; and
the power mechanism is disposed at the opening portion.

Further, a contact plate is disposed at a lower end of the opening portion;
the power mechanism is mounted on the contact plate; and
two arc-shaped grooves are further formed in the contact plate.

Further, a support is further disposed inside the soft rubber inner container;
the support includes a supporting ring and a plurality of supporting blades disposed on the supporting ring;
the supporting ring is located at the opening portion; the supporting blades are located at the main body portion; and
a plurality of hollows are formed in each of the supporting blades.

Further, the power mechanism includes a motor and an eccentric wheel;
the eccentric wheel is mounted on a rotating shaft of the motor and configured to generate a vibration; and
the eccentric wheel is semi-circular.

Further, a clamping groove is further formed in a tail of the soft rubber inner container, two buckles are disposed in the clamping groove, and a button magnet is mounted on each of the buckles and configured to be magnetically butted with a mechanism that provides push-pull power.

Further, a plurality of bamboo shoot-shaped protrusions are disposed on an inner wall of the channel of the soft rubber inner container.

With the technical solution of the present utility model, the following beneficial effects are achieved:
1. Differing from the power mechanism disposed in a middle of the soft rubber inner container to provide a vibration massage in the prior art, the power mechanism is disposed at the opening portion of the soft rubber inner container, thereby achieving a better effect.
2. The contact plate is disposed at the opening portion, and the arc-shaped grooves are formed in the contact plate to further fit the skin of the male genital organ, thereby providing a better usage experience.

### BRIEF DESCRIPTION OF DRAWINGS

FIG. 1 is a three-dimensional view of the present utility model;

FIG. 2 is a three-dimensional view of the present utility model from another perspective;

FIG. 3 is a sectional view of the present utility model;

FIG. 4 is a front view of the present utility model;

FIG. 5 is a three-dimensional view of a support in the present utility model; and

FIG. 6 is a three-dimensional view of a power mechanism in the present utility model.

In the drawings:
1. soft rubber inner container;
101. opening portion; 102. main body portion; 103. channel; 1031. bamboo shoot-shaped protrusion;
104. contact plate; 1041. arc-shaped groove;
105. support; 1051. supporting ring; 1052. supporting blade;
106. clamping groove; 107. buckle; 108. magnet;
2. power mechanism;
201. motor; and 202. eccentric wheel.

### DETAILED DESCRIPTION OF THE EMBODIMENTS

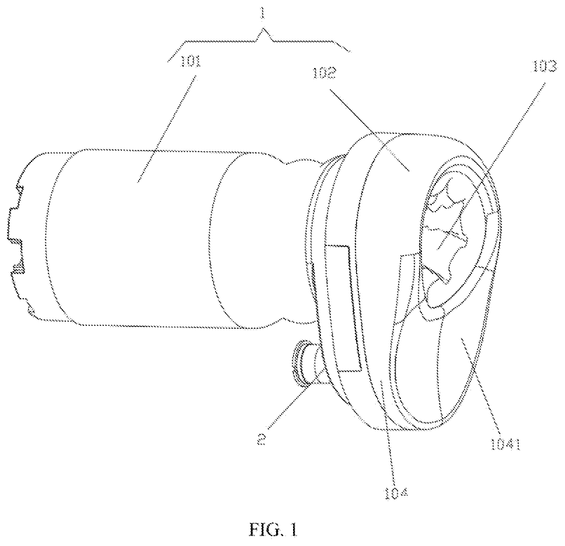

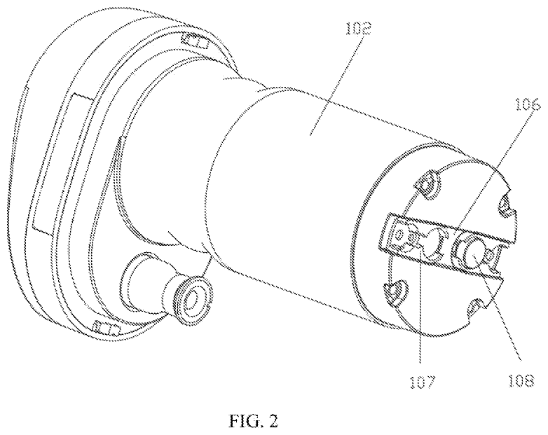

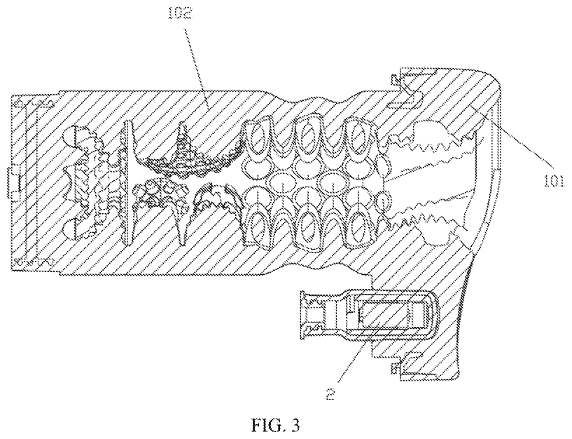

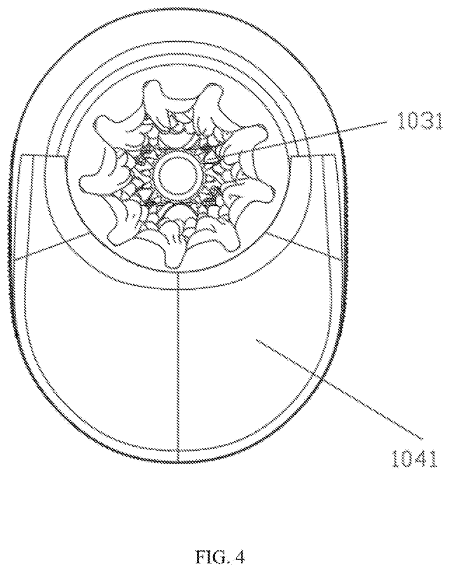

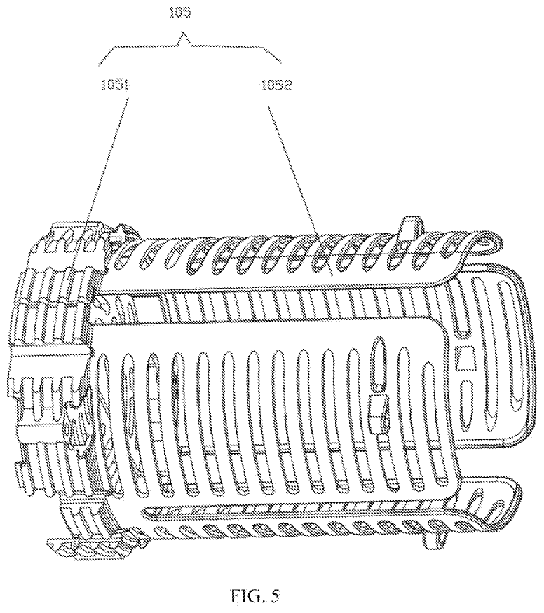

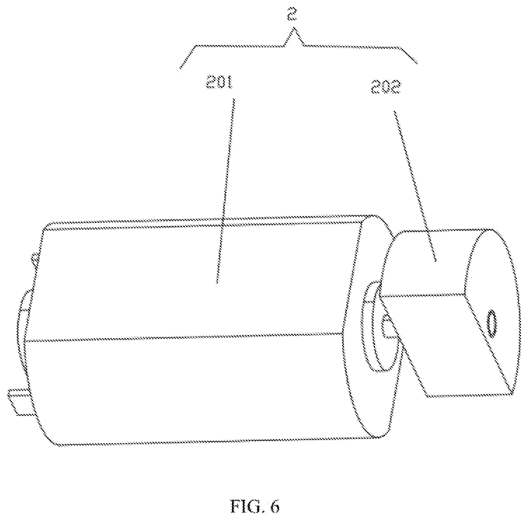

As shown in FIGS. 1 to 6, the present utility model provides an inner container opening structure of a sperm extractor, including: a soft rubber inner container 1 configured to massage a male genital organ; and a power mechanism 2 mounted on the soft rubber inner container 1 and configured to generate a vibration, where the soft rubber inner container 1 includes an opening portion 101 and a main body portion 102; the main body portion 102 is mounted at the opening portion 101, and a channel 103 is formed inside the opening portion and the main body portion; and a plurality of bamboo shoot-shaped protrusions 1031 are disposed on an inner wall of the channel 103 of the soft rubber inner container 1. The power mechanism 2 is disposed at the opening portion 101.

A contact plate 104 is disposed at a lower end of the opening portion 101; the power mechanism 2 is mounted on the contact plate 104; and two arc-shaped grooves 1041 are further formed in the contact plate 104.

A support 105 is further disposed inside the soft rubber inner container 1; the support 105 includes a supporting ring 1051 and a plurality of supporting blades 1052 disposed on the supporting ring 1051; the supporting ring 1051 is located at the opening portion 101; the supporting blades 1052 are located at the main body portion 102; and a plurality of hollows are formed in each of the supporting blades 1052.

The power mechanism 2 includes a motor 201 and an eccentric wheel 202; the eccentric wheel 202 is mounted on a rotating shaft of the motor 201 and configured to generate a vibration; and the eccentric wheel 202 is semi-circular.

A clamping groove 106 is further formed in a tail of the soft rubber inner container 1, two buckles 107 are disposed in the clamping groove 106, and a button magnet 108 is mounted on each of the buckles 107 and configured to be magnetically butted with a mechanism that provides push-pull power.

The principle of the present utility model is as follows:

The soft rubber inner container 1 includes an opening portion 101 and a main body portion 102; the main body portion 102 is mounted at the opening portion 101, and a channel 103 is formed inside the opening portion and the main body portion to accommodate the male genital organ for massage and sperm extraction; and
a contact plate 104 is disposed at a lower end of the opening portion 101; the power mechanism 2 is mounted on the contact plate 104 and configured to generate a vibration for massage; and two arc-shaped grooves 1041 are further formed in the contact plate 104 to further fit the skin of the male genital organ.

The above are only preferred embodiments of the present utility model and are not intended to limit the scope of patent of the present utility model. Any equivalent structural changes made using the contents of the specification and drawings of the present utility model under the concept of the present utility model, directly/indirectly applied in other related technical fields, are included within the scope of patent protection of the present utility model.

What is claimed is:

1. An inner container opening structure of a sperm extractor, comprising:
a soft rubber inner container configured to massage a male genital organ; and
a power mechanism mounted on the soft rubber inner container and configured to generate a vibration,
wherein the soft rubber inner container comprises an opening portion and a main body portion; the main body portion is mounted at the opening portion, and a channel is formed inside the opening portion and the main body portion; and
the power mechanism is disposed at the opening portion.

2. The inner container opening structure of a sperm extractor according to claim 1, wherein a contact plate is disposed at a lower end of the opening portion;
the power mechanism is mounted on the contact plate; and
two arc-shaped grooves are further formed in the contact plate.

3. The inner container opening structure of a sperm extractor according to claim 1, wherein a support is further disposed inside the soft rubber inner container;
the support comprises a supporting ring and a plurality of supporting blades disposed on the supporting ring;
the supporting ring is located at the opening portion; the supporting blades are located at the main body portion; and
a plurality of hollows are formed in each of the supporting blades.

4. The inner container opening structure of a sperm extractor according to claim 1, wherein the power mechanism comprises a motor and an eccentric wheel;
the eccentric wheel is mounted on a rotating shaft of the motor and configured to generate a vibration; and
the eccentric wheel is semi-circular.

5. The inner container opening structure of a sperm extractor according to claim 1, wherein a clamping groove is further formed in a tail of the soft rubber inner container, two buckles are disposed in the clamping groove, and a button magnet is mounted on each of the buckles.

6. The inner container opening structure of a sperm extractor according to claim 1, wherein a plurality of bamboo shoot-shaped protrusions are disposed on an inner wall of the channel of the soft rubber inner container.
